(19) United States
(12) Patent Application Publication
(10) Pub. No.: US 2020/0327988 A1
(43) Pub. Date: Oct. 15, 2020

(54) TECHNIQUES FOR CONTEXT-DRIVEN MEDICAL CLAIM PROCESSING TO IDENTIFY MEDICAL CLAIMS OF INTEREST AND A CLINICAL CLAIM SURVEILLANCE SYSTEM IMPLEMENTING SAME

(71) Applicant: ADVANCED MEDICAL STRATEGIES, LLC, Lynnfield, MA (US)

(72) Inventors: Peter BORANS, Waxhaw, NC (US); Stacy BORANS, Chalfont, PA (US); Jon DONOGHUE, Amherst, NH (US)

(21) Appl. No.: 16/844,445

(22) Filed: Apr. 9, 2020

Related U.S. Application Data
(60) Provisional application No. 62/831,253, filed on Apr. 9, 2019.

(51) Int. Cl. G16H 50/20; G06Q 10/10; G16H 70/20; G16H 70/40; G06Q 40/08; G06F 16/2455

(52) U.S. Cl. CPC G16H 50/20; G06Q 10/10; G06F 16/24564; G16H 70/40; G06Q 40/08; G16H 70/20

(57) ABSTRACT

In general, techniques for context-driven medical claim (CDMC) processing and a system implementing same are disclosed herein to enable N number of different payer entities (PEs) to receive and process medical claims and identify medical claims of interest, e.g., medical claims with at least one predetermined abnormality, preferably before adjudication and payment. In an embodiment, a pipeline implementing CDMC techniques provides a rule-based engine to detect predetermined claim abnormalities based on, for instance, rules comprising logic that, when instantiated, compares target line items/components of a medical claim against data within medical data repositories (e.g., diagnosis and treatment databases, patient outcome data, and pharmaceutical databases), and/or detects the presence of a predetermined claim abnormality based on trainable medical model(s) or other machine-learning approaches.

### CROSS-REFERENCE TO RELATED APPLICATIONS

This application claims the benefit of U.S. Provisional Patent Application Ser. No. 62/831,253, filed on Apr. 9, 2019 which is fully incorporated herein by reference.

### TECHNICAL FIELD

The following disclosure relates generally to systems for electronic medical claims processing, and more particularly, to techniques for context-driven medical claim processing to identify medical claims of interest for secondary review by an associated payer entity (PE) and a claim surveillance system implementing same.

### BACKGROUND

Electronic processing and adjudication of medical claims (also referred to as automated processing) presently accounts for upwards of 90% of all medical claims in the United States. The number of manually-processed claims continues to decrease each year, and the present trends suggest that in the near future virtually 100% of medical claim processing and adjudications will include at least some automated processing.

However, present approaches to automated processing of medical claims focus on generally two aspects of claim processing, namely claim acquisition and post-adjudication auditing and investigations. Payer entities (PEs) such as insurance providers and Third Party Administrators (TPAs) utilize automated claim acquisition to allow for providers, e.g., hospitals, doctors, and mental health professionals, to submit medical claims in an electronic format. Following acquisition, adjudication in the form of a denial (or partial-denial) and/or payments from the PEs to the providers based on the submitted medical claims occurs automatically, or based on manual review. Automated post-adjudication auditing and investigations occur based on, for instance, a predetermined audit schedule (e.g., weekly, monthly, yearly) and during claw back actions to recover payments connected with fraud or error. However, such automated post-adjudication auditing and investigations tend to operate on data generated during adjudication of a claim, e.g., accounting/financial data, rather than directly on content of the claims themselves.

Automated processing of medical claims beyond claim acquisition and post-adjudication auditing and investigations raises numerous non-trivial challenges.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are enlarged and positioned to improve drawing legibility. Further, the particular shapes of the elements as drawn, are not intended to convey any information regarding the actual shape of the particular elements, and have been solely selected for ease of recognition in the drawings.

FIG. 1 depicts a block diagram of an exemplary network environment and Clinical Claim Surveillance (CCS) computing system in accordance with an embodiment of techniques described herein, as well as various computing systems associated with one or more client users of the CCS system.

FIG. 2 depicts a block diagram of an additional exemplary network usage environment in accordance with an embodiment of techniques described herein.

FIG. 3 depicts a process flow suitable for use by the Clinical Claim Surveillance system of FIG. 1, in accordance with an embodiment of the present disclosure.

FIG. 4 depicts a context-driven medical claim (CDMC) processing pipeline suitable for use in the Clinical Claim Surveillance system of FIG. 1.

FIGS. 5A-5B show an example method for identifying one or more predetermined claim abnormalities in a received medical claim, in accordance with embodiments disclosed herein.

FIG. 6 is a block diagram illustrating component-level functionality provided by a plurality of electronic circuits that, when in combined operation, are suitable for performing and configured to perform at least some of the techniques described herein.

### DETAILED DESCRIPTION

As discussed above, automated processing of medical claims beyond claim acquisition and post-adjudication auditing and investigations raises numerous non-trivial challenges. These challenges are due, at least in part, to the continued use of medical claim form standards and terminology whose adoption pre-dates computing systems. Various pharmaceutical and/or medical treatments are codified and represented via, for instance, a series of letters and/or numbers. Likewise, similar codes to indicate a diagnosis, pharmaceutical dosage, treatment duration, and other treatment-related parameters appear in medical claim line items rather than full-text descriptions. Providers tend to verify accuracy of the various codes through internal checks and safeguards, but with upwards of tens of thousands of various codes (and code combinations), errors in claims submitted to payer entities (PEs) remain inevitable.

Thus, PEs face numerous difficulties when attempting to identify errors/abnormalities in medical claims before adjudication occurs, and importantly, before treatment is authorized and/or payments are disbursed. Patent errors, e.g., errors that appear clearly on the face of a medical claim, can be detected by careful manual (and sometimes automated) review by PEs. Some such example so-called “patent” claim errors manifest as missing/incomplete claimant or provider information, invalid and/or missing codes, and unreadable text. However, latent errors in medical claims remain essentially undetectable until after, for instance, post-adjudication auditing to identify abnormally high claim payouts. Such latent errors can include, for instance, a mismatch between diagnosis and prescribed therapy/treatment, a mismatch between patient age and treatment/drug, incorrect treatment sequence (e.g., chemotherapy drug B ordered before a prerequisite drug A was administered), incompatible drugs (e.g., two or more prescribed drugs that have known/dangerous interactions and/or increased risk of complications), and an improper treatment duration/frequency. Still further, latent errors can include, for instance, inappropriate use of pharmaceutical drug based on, for instance, the drug being used to treat a disease/condition in an off-label manner, e.g., not recognized by the Federal Drug Agency (FDA), and treatment that utilizes expensive drugs known as orphan drugs (e.g., designed to treat rare diseases/conditions that afflict less than 250 k people within the United States) when less-expensive and medically-effective alternatives are available. Still further, various costs such as implant pricing remains relatively opaque to PEs, and often latent claim errors include line items for implant devices that, in view of regional and/or national pricing data, could be satisfied by an alternative implant or otherwise purchased for less cost from a different supplier and/or through re-negotiated pricing with suppliers.

Detecting the presence of such patent and latent claim errors (referred to herein collectively and without limitation as claim abnormalities or simply abnormalities) in submitted medical claims amounts to a proverbial needle in a haystack as submitted medical claims include the above-mentioned complex coding schemes, and such submitted claims number in the upwards of tens of thousands per year for each PE. Unfortunately, the relatively small percentage of claims with undetected abnormalities account for a disproportionate amount of avoidable waste in both cost and inappropriate/unnecessary medical treatments.

Still further, each PE faces different and distinct challenges with regard to the types of abnormalities that occur specific to their clients/providers as well as challenges related to contractual and/or legal obligations to adjudicate claims in a timely manner. For instance, some PEs have a substantial number of elderly claimants (and/or claimants with rare and/or uncommon conditions and diseases) relative to other PEs. Therefore, the particular claim abnormalities of concern for each PE can include common abnormalities (e.g., abnormalities that virtually all PEs will encounter that can cause the above-discussed waste) as well as PE-specific abnormalities and/or conditions related to their specific provider/claimant group.

In view of the foregoing, real-time automated processing of medical claims to intelligently detect and flag claims with abnormalities (preferably before adjudication) utilizing a wide-range of disparate PE-provided criteria remains a significant impediment to continued development and adoption of robust automated medical claim processing.

Thus, in general, techniques for context-driven medical claim (CDMC) processing and a system implementing the same are disclosed herein to enable N number of different payer entities (PEs) to receive and process medical claims, and identify medical claims of interest, e.g., medical claims with at least one predetermined abnormality, preferably before claim adjudication that ultimately leads to payment by a PE and/or authorization of care (e.g., services, medical devices, pharmaceutical therapies).

In an embodiment, a pipeline implementing CDMC techniques provides a rule-based engine to detect predetermined claim abnormalities based on, for instance, rules comprising logic that, when instantiated by a controller, compares target line items/components of a medical claim against parameters within, and/or derived from, medical data repositories (e.g., diagnosis and treatment databases, patient outcome data, and pharmaceutical databases). Alternatively, or in addition, the rules-based engine detects the presence of a predetermined claim abnormality based on trainable medical model(s) or other machine-learning approaches, heuristics, and/or a combination thereof.

Further, the CMDC pipeline may be implemented in a distributed network context to allow for at least a portion of the pipeline to be executed on one or more different computer systems, and preferably, one or more computer systems provided or otherwise managed by PEs. The PEs may therefore collectively interact/communicate with a common (or shared) system implementing the CMDC pipeline (e.g., via cloud-based services). Accordingly, various data security and Health Insurance Portability and Accountability Act (HIPAA)-concerns can be minimized or otherwise mitigated by ensuring that, for example, one or more stages of the CDMC pipeline offload or otherwise initiate collection and processing of sensitive claimant data (e.g., medical history, claimant information such as address, data of birth, gender) on PE-controlled servers/instances. Further, secondary review can be initiated on the flagged claims as part of the pipeline and be performed on, for instance, computer systems owned or managed by the associated PE.

In one specific non-limiting example embodiment, a system for processing medical claims implements a CDMC pipeline consistent with the present disclosure. The system can be configured to receive medical claim requests from a plurality of provider entities to identify medical claims of interest for secondary review by an associated PE. Likewise, the system can be configured to provide medical claim processing for a plurality of different PEs. In this embodiment, the system includes a controller communicatively coupled to a computer network, with the computer network allowing for multiple PEs, provider entities, and/or other authorized users to communicate with the system, for example. In addition, the controller implements the CDMC pipeline to allow for submission of medical claims for abnormality detection and reporting. In particular, the controller can be configured receive a medical claim request via the computer network, the received medical claim request being associated with a target PE. The controller then selects at least a first claim rule from a plurality of claim rules based at least in part on the target PE and the received medical claim request, the first claim rule including predetermined logic to detect a first predetermined claim abnormality. The controller then identifies the received medical claim request as a claim of interest based on instantiating the predetermined logic of the first claim rule and detecting the predetermined abnormality as present in the received medical claim. The controller may then store an indicator of the received medical claim request in a memory in response to identifying the received medical claim request as a claim of interest.

Thus, users associated with the PEs, providers, or other authorized entities may then communicate/interact with the system (e.g., via a wide area network such as the Internet) and initiate/execute processes as variously disclosed herein such as submitted claims and monitoring claim status, and to perform various other tasks such as retrieval of medical claims of interest identified by the system for secondary review.

One or more embodiments described herein also enable one or more processor-based computing systems to perform automated claim processing operations to facilitate the retrieval, analysis, identification, and reporting of medical claims, such as (by non-limiting example) insurance-related or other claims for payment and/or reimbursement regarding one or more medical and/or pharmaceutical treatments (e.g., by implementing a CDMC pipeline consistent with the present disclosure). In this manner, the described techniques may enable the identification of particular such medical claims by both PE claim managers and providers/claimants related to those medical claims, such as by enabling the automated identification of anomalous or problematic (also referred to herein as abnormal) medical claims, as well as additional operations detailed elsewhere herein. In an embodiment, some or all of the techniques may be performed by one or more computing systems operating as a Clinical Claim Surveillance (“CCS”) system as discussed herein. Moreover, functionality described herein with respect to such a CCS system may be provided by one or more networked computing systems, such networked computing systems operated by one or both of a clinical claim surveillance service provider operating the CCS system and a local client application associated with the CCS system but executing on client devices of its users.

In certain embodiments, a CCS system implementing a CDMC pipeline consistent with the present disclosure may retrieve or otherwise be provided with communications representative of one or more medical claims, such as by a clinical claim manager client (or PE manager) of the CCS system, in order to identify one or more anomalous or problematic conditions presented by a medical claim. For example, the CCS system may determine one or more inappropriate drug combinations when performing rule-based analysis of a medical claim that includes indications of a plurality of prescribed drugs administered to a claimant as part of medical and/or pharmaceutical treatment. As another example, the CCS system may identify an unapproved usage of a prescribed drug indicated by such a clinical claim, such as if a specialty pharmaceutical has been used “off-label” in the clinical claim (i.e., used in a manner that has not been approved for the specialty pharmaceutical by the Food & Drug Agency or other relevant entity). As still another example, the CCS system may identify an inappropriate use of one or more pharmaceuticals or medical treatments indicated by a clinical claim submitted to the CCS system based on information specific to a specified claimant, such as if a medical treatment indicated as being suitable only for adults has been provided to a minor or other at-risk patient type. In this manner, the CCS system may identify and possibly prevent hazardous conditions associated with particular medical and/or pharmaceutical treatments and treatment combinations, as well as significantly reduce fraud, waste, and abuse associated with such treatments and treatment combinations.

As used herein, the term “medical claim,” or simply “claim,” refers to any physical document, electronic document, or data structure indicative of or representing one or more medical and/or pharmaceutical treatments performed on, for, or on behalf of an individual. However, in the context of automated processing of claims, such as via a CDMC pipeline consistent with the present disclosure, the term medical claim refers to an electronic/digital representation of a medical claim (which may, or may not also have a physical or “paper” counterpart) unless otherwise noted herein. Medical claims may also be referred to herein as clinical claims.

The term “claimant,” also referred to herein as a “claimant user,” refers generally to a patient or individual that through receiving medical care, drugs, therapies, and/or medical devices from a provider, caused the submission of a medical claim to an associated PE. In some scenarios, claimant user may also refer to a provider or a provider-authorized user who has the privileges to submit medical claims to a PE and monitor status thereof.

As generally referred to herein, a PE refers to any entity that has a fiducial and/or contractual responsibility to adjudicate and pay medical claims submitted by provider entities. Example non-limiting PE entities include Third Party Administrators (TPAs) and insurance providers.

A provider entity, also referred to herein as simply a provider, refers to any entity who provides medical services including, without limitation, therapeutic services (e.g., rehabilitation, mental health counseling), in-patient diagnosis and treatment of diseases and conditions (e.g., within a hospital setting, addiction clinic, hospice care), emergency medical care (e.g., ambulance services, emergency rooms), medical devices, and/or pharmaceutical drugs. Thus, the term provider entity refers generally to any entity having the contractual and/or legally-recognized right to submit medical claims to a PE for payment and/or medical treatment authorization purposes.

Reference herein to a “user” or “users” without further designation may include any individuals or entities interacting in various scenarios with an embodiment of the CCS system, and may include past, future or current such users.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In at least certain embodiments, the CCS system may store, maintain, modify, apply, and otherwise utilize one or more sets of rules associated with particular medical treatments, pharmaceutical or other substances, or other aspects of clinical claims provided to (or potentially provided to) a CCS system. Such rules may include, as non-limiting examples, rules regarding appropriate and/or inappropriate medical procedures and/or other treatments, pharmaceutical usage, pharmaceutical combinations, regulatory approvals, regulatory application histories, medical histories, clinical histories, sexual histories, clinical trial information, historical research information, patient historical information, claimant records, financial information, or other rules related to itemized and/or combinatorial aspects of clinical claims.

In various embodiments, such rules may be applied individually or (more typically) in combination to identify conditions specific to one or more clinical claims provided to the CCS system for analysis. As will be appreciated, a combined application of even a few hundred such rules may result in the CCS system being enabled to identify tens of thousands (or more) such conditions that may not be readily identifiable without technological improvements provided as part of the CCS system to one or more computing systems executing the CCS system.

In certain embodiments, the CCS system may determine to apply the entirety of its medical and pharmaceutical rules database to each clinical claim received by the CCS system for analysis and identification. In other embodiments, the CCS system may determine to apply only a subset of such medical and pharmaceutical rules to a particular clinical claim, such as in accordance with policies and or user preferences associated with a particular provider entity submitting the clinical claim and/or PE receiving the claim, in accordance with data specific to a claimant or claimant category associated with the clinical claim, in accordance with the information regarding insurance or other coverage associated with the clinical claim. In this embodiment, such claim pre-conditions are also referred to herein as rule pre-triggers. Rule pre-triggers may be created or otherwise modified by PEs based on PE-provided parameters, similar to the PE-provided parameters (also referred to herein as PE-provided claim rule parameters) utilized during rule processing stages as discussed herein.

In some embodiments, medical claims submitted for analysis and identification via the CCS system, such as by a PE manager or other party associated with a client entity of a service provider operating the CCS system, may include various input data associated with such medical claims. As non-limiting examples, such input data may include current and historical claimant medical information, current and/or historical claimant financial information, dates of service, diagnostic information, medical procedure codes, pharmaceutical codes, approved indications associated with a pharmaceutical, non-approved indications associated with a pharmaceutical, cost information associated with a pharmaceutical (e.g., Medicare payment information and/or reasonable associated free-market costs), dosage information associated with a pharmaceutical, medical comments associated with the pharmaceutical, insurance policy information, insurance coverage information, claimant employer information or information regarding other claimant-associated entities, financial coverage information, claimant family history information, claimant identification codes, pharmaceutical trade name information, pharmaceutical generic name information, pharmaceutical categorizations, and other related information. Diagnostic information may include, but is not limited to, diagnosis codes such as the International Classification of Diseases (ICD) codes published by the World Health Organization (WHO), medical diseases/syndromes, medical signs, medical symptoms, abnormal medical findings, medical complaints, social circumstances, etc.

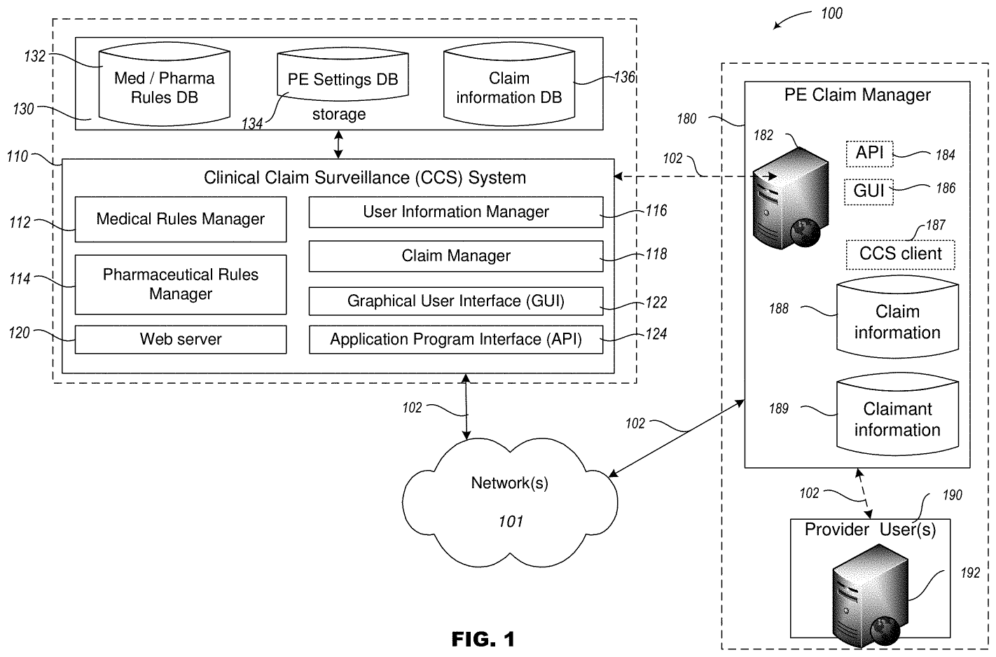

Turning to the figures, FIG. 1 depicts a block diagram of an exemplary networked environment 100 that includes a Clinical Claim Surveillance (CCS) system 110, as well as various computing systems associated with one or more client claim manager users (also referred to herein as PE managers) and/or with one or more claimant users of the CCS system (also referred to herein as provider users).

The illustrated example of FIG. 1 includes a number of PE manager computing systems 180 and a number of provider user computing systems 190 that are each interacting at various times with the CCS system 110 via one or more intervening networks 101. Each of the PE manager computing systems 180 may also be referred to herein as PE instances. Likewise, each of the provider user computer systems 190 may also be referred to herein as provider client computers.

The interactions of PE manager computing systems 180, provider user computing systems 190, and other entities within the CCS system 110 may occur in various ways, such as in an interactive manner via one or more graphical user interfaces 122 that are provided by the CCS system 110 to those users and/or other entities via at least some Web pages of a CCS system Web site provided by Web server 120. In addition, interactions with various components of the CCS system 110 may be through a so-called “app” executed on a remote computing device such as a smart phone or laptop computer. The PE manager computing systems 180 can include, for instance, API 184, GUI 186, and CCS client 187 for accessing and interacting with the CCS system 110.

In addition to or in conjunction with such interactions, interactions with the CCS system 110 may occur in a programmatic manner by one or more client software applications via an Application Program Interface (API) 124 provided by the CCS system 110 that allows computing systems and/or programs to invoke various functionality programmatically, such as using Web services or other network communication protocols.

In the illustrated embodiment, various interactions between the PE manager computing systems 180 and the CCS system 110 may be performed using either or both of a web browser 182 and a CCS application or “app” 187 executing on, for instance, one or more of the PE manager computing systems 180. Such interactions can be based at least in part on claim information 188 and/or claimant information 189, such as may be retrieved and/or stored by the PE manager computing systems 180. Similarly, various interactions between provider computing systems 190 and the CCS system 110 may be performed using either or both of a web browser 192 and a CCS application (not shown) executing on the provider computing system 190. Each of the PE manager computing systems 180 and the provider computing systems 190 may comprise a fixed computing system or a mobile computing device, as detailed elsewhere herein.

In the illustrated embodiment, the CCS system 110 includes a medical rules manager component 112; a pharmaceutical rules manager component 114; a user information manager component 116; and a claim manager component 118. In addition, the CCS system 110 includes the aforementioned Web server 120, GUI 122, and API 124, all of which may facilitate various interactions with any or all of the PE manager computing systems 180 and provider computing systems 190.

As further shown, the CCS system 110 is communicatively coupled to storage components 130. The storage components comprise, for example, a medical and/or pharmaceutical rules database (DB) 132, a PE settings DB 134, and claim information DB 136. Preferably the storage components 130 get provided by a third-party data storage service provider such as Amazon Web Services (AWS) offered by Amazon Web Services, Inc., and Microsoft Azure offered by Microsoft, Inc., or any other suitable provider of various network- and/or cloud-based storage services. Alternatively, or in addition, storage components 130 may be integrated into the CCS system directly, and may be operated by a CCS service provider that operates the CCS system 110.

In scenarios in which the CCS system 110 provides one or more Web sites via Web servers 120, at least some PE manager users and provider users (not shown) perform authenticated interactions and execute various functions and processes of the CS system 110 via a client computing device as disclosed herein. Such client computing device include, for example, the PE manager computing system 180 and/or provider computing system 190. In addition, such client computing devices may also include any computer device capable of communicating with the CCS system 110 over networks 101, such as to obtain Web pages or other electronic information pages from the CCS system, and to optionally provide various information related to one or more clinical claims and/or manage workflows related to one or more clinical claims.

In any such cases, such users may access one or more Web sites provided by the CCS system to obtain one or more Web pages, such as to view information about, search for, browse for, and/or provide information related to clinical claims, client claim managers, and/or claimants. The CCS system 110 can include a user database (not shown) to store user names, passwords, and associated rights and privileges to ensure that data access is limited only to authorized system areas, and preferably, in a minimum-privilege scheme to ensure that a given user has the minimum privilege levels and rights to perform their particular role within the CCS system 110.

Accordingly, the CCS system 110 stores various types of user information to enable such interactions, and further information about all interactions and activities performed by the user with respect to the CCS system for audit and tracking purposes.

The network 101 comprises a wide-area network (WAN) such as the Internet, although in other embodiments the network 101 may have other forms including private networks, and/or a combination of public and private networks. For example, the network 101 may instead be a private network, such as a corporate or university network that is wholly or partially inaccessible to non-privileged users. Further, the network 101 can collectively comprise private and public networks, with one or more of the private networks having access to and/or from one or more of the public networks, and vice-versa.

Furthermore, the network 101 can include various types of wired and/or wireless networks depending on a desired configuration. In addition, and as shown in FIG. 1, users of the PE manager computing systems 180 and provider computing systems 190 may use such computing systems and/or other client devices (not shown) to interact with the CCS system 110 to obtain various described functionality via the network 101, and in doing so may also provide various types of information to the CCS system 110. Moreover, users of the networked environment 100 may interact with the CCS system 110 and/or one or more other users and/or service providers using an optional private or dedicated connection, such as a dedicated connection 102. For example, the dedicated connection 102 can include one or more VPN (Virtual Private Network) connections.

Furthermore, the CCS system 110 can receive and provide various types of information from additional information provider computing systems, such as those that may be operated by government agencies, non-client insurance providers, or other certifying authorities (not shown). The CCS system 110 may optionally retrieve various information from such information provider computing systems, such as one or more exemplary or other representative medical claims to utilize and/or analyze on behalf of users of the CCS system 110. In addition, the CCS system 110 may provide identified medical claims (also referred to herein as medical claims of interest) to such information providers, such as to provide such medical claims as evidence of compliance with regulations or policies promulgated by clients of the CCS system 110 or other entities.

Continuing on, the PE claim manager users (e.g., users who access the CCS system 110 via rights and privileges established via PE manager computing systems 180) can perform a plurality of activities related to medical claims via interactions with CCS system 110. As non-limiting examples, such interactions include receiving information associated with a PE manager user by the CCS system 110, such as for inclusion within one or more medical claims; receiving and handling requests from provider users, such as to identify one or more PE managers associated with a medical claim based on specified criteria, to request documentation or other information regarding a specified medical claim; and other authorized interactions.

In addition, the CCS system 110 can initiate various operations regarding either or both of the PE manager users and provider users independent of interactions with those users themselves. Non-limiting examples of such operations include storage and analysis of information regarding such users for current and future use, including storing, tracking and analyzing information related to interactions by those users with the CCS system 110 or with other users; providing notifications to users of the CCS system 110 regarding medical claims that may be of interest to such users based on user characteristics, user preferences, user-provided criteria, or previous interactions with the CCS system 110. In addition, the CCS system 110 can provide medical claim identification information (also referred to herein as an identifier of a medical claim) or other analytical information regarding various aspects of transactions performed regarding medical claims identified by or otherwise analyzed via the CCS system 110.

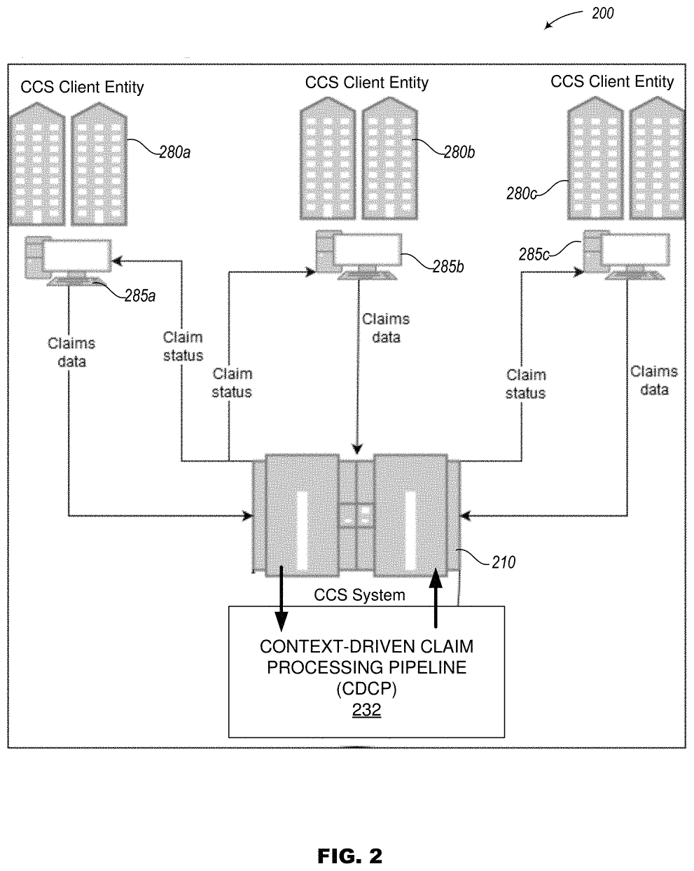

FIG. 2 provides a high-level block diagram of an additional exemplary network usage environment 200 in accordance with an embodiment of techniques described herein. In the depicted environment, multiple client entities 280a, 280b, and 280c (collectively, CCS client entities 280) are communicatively connected to a CCS system 210 via one or more respective networked client claim manager computing systems 285a, 285b, and 285c (collectively, client claim manager computing systems 285). Each CCS client of the CCS client entities can comprise a PE, provider, or other authorized client entity.

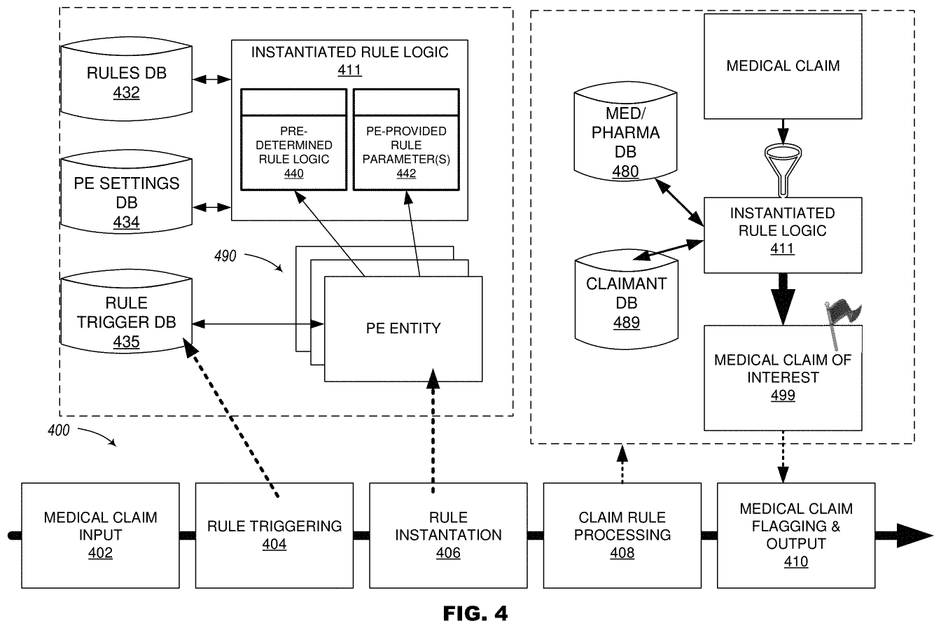

As further shown, the CCS system 210 implements a context-driven claim processing (CDCP) pipeline 232. In an embodiment, the context-driven claim processing pipeline 232 is collectively implemented by various components of the CCS system 110, e.g., medical rules manager component 112, pharmaceutical rules manager components 114 in combination with the additional components such as, for instance, storage components 130, claim information DB 188, and claimant information DB 189. The CDCP pipeline 232 is configured to receive medical claims data, referred to herein as also simply medical claims, and instantiate claim processing logic comports with PE-defined criteria. One such example CDCP pipeline is discussed in greater detail below with regard to FIG. 4.

In operation, medical claims data is provided to the CCS system 110 by the CCS client entities via the client claim manager computing systems 285. The medical claims data can include one or more medical claims and associated information including, for example, claimant/patient information (address, phone, date of birth, gender, race), provider information, and an associated PE for the claim. In turn, the CCS system 210 provides the medical claims data to an input stage of a CDCP pipeline 232, which then processes the received medical claims via the instantiated processing logic based on the associated PE. The CDCP pipeline 232 then utilizes various components and data stores/databases local to the CCS system 110, or otherwise available via network 100, to identify claims of interest based on the received medical claims having one or more predetermined abnormalities.

In an embodiment, the CCS system 110 then provides one or more indications of at least the claims of interest identified by the CDCP pipeline 232. The one or more conditions can include the CCS system 110 providing output data regarding the status of such medical claims to the relevant PE manager computing system and/or to the relevant provider user computing system. Alternatively, or in addition, the CCS system 110 provides one or more indications regarding one or more clinical claims provided to the CCS system 110 that are not associated with any identified abnormalities and/or problematic conditions, such as to indicate one or more subsets of medical and/or pharmaceutical rules that have been applied to those clinical claims in reaching the determination.

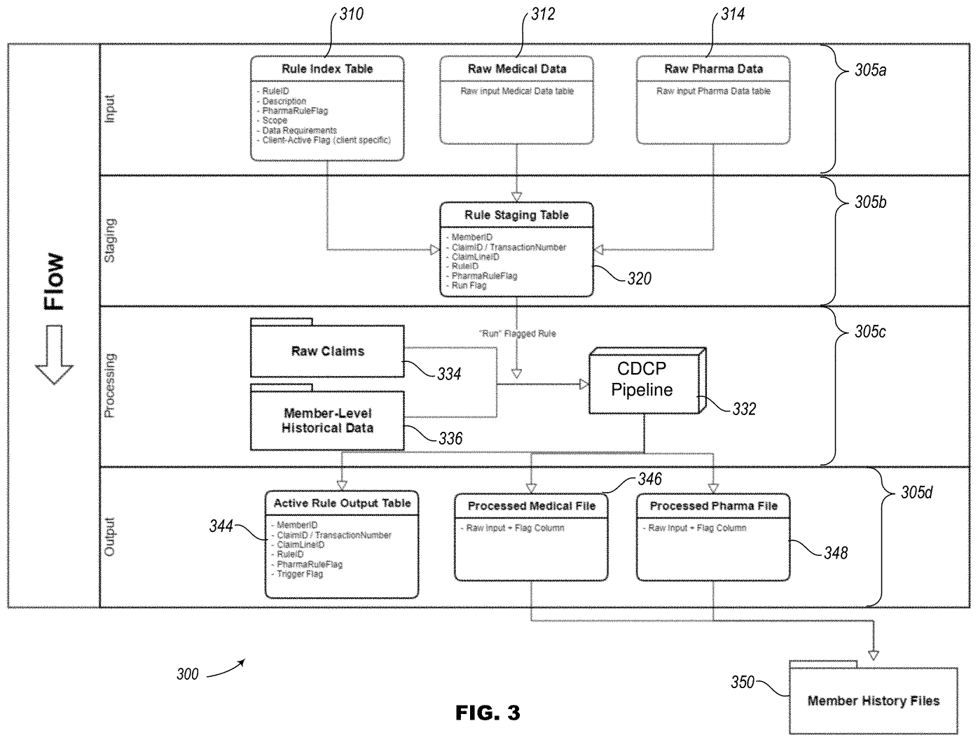

FIG. 3 depicts a process flow 300 for a Clinical Claim Surveillance system in accordance with an embodiment of the present disclosure. As shown, the process flow 300 comprises four distinct stages of operation. In particular, the process flow comprises an input stage 305a, a staging operations 305b, a clinical claim processing 305c, and an output stage 305d.

The input stage 305a depicts input data generally received by the CCS system 110 prior to receiving information specific to any particular medical claim. In particular, in input stage 305a the CCS system 110 receives a rule index table 310 that in the depicted embodiment includes a claim rule identifier (also referred to herein as a rule identifier) associated with each claim rule, a claim rule description, one or more pharmaceutical rule flags, a rule scope (indicative of clinical claim criteria for clinical claims to which the particular rule may be appropriately applied), data requirements (indicative of clinical claim data required for application of the particular rule), and optional PE-active flags (to indicate one or more PE associated with application of the rule, such as if particular PEs of the CCS system 110 have opted in or out of the particular rule being applied to clinical claims submitted by that client). Note, the input stage 305a may also utilize a lookup table that associates each PE with one or more of the aforementioned claim rules. In this example, the PE-active flag may simply be a Boolean value that indicates whether a given rule associated within a PE is enabled or disabled.

The input stage 305a further includes raw medical data 312 and raw pharmaceutical data 314. In an embodiment, input medical data may be comprised of multiple pieces of information, non-limiting instances of which may include: member identifier, claim identifier and/or other information, diagnoses, procedure details, dates of service, provider identifier and/or other information, network and costs. Input pharmaceutical data may be comprised of multiple pieces of information, including but not limited to member identifier, claim identifier and/or other information, drug types and quantities, dates of service, provider identifier and/or other information, network and costs.

As further shown in FIG. 3, the CCS system 110 performs staging operations 305b in order to generate rule staging table 320 by integrating information provided during input stage 305a as part of the rule index table 310, the raw medical data 312, and the raw pharmaceutical data 314. In the particular example of FIG. 3, the resulting rule staging table 320 includes a claimant-specific member identifier, a claim-specific identifier, one or more line identifiers to identify a portion or portions of a submitted medical claim to which each relevant rule has been applied and/or flagged, a rule identifier (such as to identify the particular rule that has been satisfied by the identified portion of the medical claim), a pharmaceutical rule flag, and a “run flag,” to identify why the particular rule is to be applied to particular medical claims and/or specified portions of each medical claim satisfying various criteria associated with that rule.

During clinical claim processing 305c, the CCS system 110 uses the generated rule staging table 320 to apply each flagged rule via CDCP pipeline 332 to a clinical claim received by the CCS system. The CDCP pipeline 332 may be implemented as the CDCP pipeline 232 as discussed above, the description which will not be repeated for brevity. Also note, the CDCP pipeline 232 may be implemented as the example CDCP pipeline discussed below with regard to FIG. 4. The CCS system 110 then applies, via CDCP pipeline 332, each enabled claim rule to raw clinical claim data 334 based on the raw claim data within the medical claim itself and the PE associated with the claim, as well as on any claimant-specific historical data 336 provided in association with the medical claim. Note, the CDCP pipeline 332 may also be accurately referred to herein as a claim rule engine, or simply a rule engine.

Continuing with FIG. 3, in output stage 305d the CCS system 110 provides segmented output data via the CDCP pipeline 232. In particular, the CDCP pipeline 332 generates active rule output table 344 to provide diagnostic information regarding the application of one or more claim rules to the relevant input medical claim data. As shown, active rule output table 344 includes a claimant-specific member identifier associated with the analyzed medical claim, a claim-specific identifier associated with the analyzed clinical claim, one or more line identifiers to identify a portion or portions of a submitted medical claim that has satisfied the identified rule, a rule identifier (such as to identify the particular rule that has been satisfied by the identified portion of the medical claim), a pharmaceutical rule flag, and a “trigger flag,” to identify why the particular rule has been satisfied by the identified medical claim and/or specified portion(s) of that medical claim.

In addition to the active rule output table 344 generated by the CDCP pipeline 332, the CCS system 110 outputs information indicative of one or both of a processed medical claim file 346 (such as may include both the raw input data of the relevant medical claim as well as an associated column of rules satisfied by that claim) and a processed pharmaceutical medical claim file 348. The processed pharmaceutical claim 348 can include, for example, both the raw input data of the relevant pharmaceutical portions of a medical claim as well as an associated column of pharmaceutical-related rules satisfied by that medical claim.

Also shown in FIG. 3, one or both of the processed medical clinical claim file 346 and processed pharmaceutical clinical claim file 348 are used to modify one or more claimant history files 350 associated with each processed medical claim. For example, in various embodiments, the CCS system 110 may modify one or more claimant history records internal to the CCS system 110, such as may be used in future rule processing; one or more claimant history records stored by the client, such as may be used to optionally store results from the CCS system 110 regarding rule execution, for example.

FIG. 4 shows one example context-driven claim processing (CDCP) pipeline 400 suitable for use as the CDCP pipeline 232/332 in the CCS system 110 discussed above. As shown, the CDCP pipeline 400 includes a plurality of stages including a medical claim input stage 402 (also referred to herein as an input stage), a rule triggering stage 404, a rule instantiation stage 406, a claim rule processing stage 408, and a medical claim flagging and output stage 410 (also referred to herein as an output stage). Each stage will now be discussed in turn.

The input stage 402 is configured to receive at least one medical claim, which may be referred to also as an unprocessed medical claim(s). Note, in some scenarios the CDCP pipeline 400 is dedicated to processing of medical claims for a single PE. The CCS system 110 may therefore instantiate multiple CDCP pipelines 400 (also referred to as PE pipeline instances) to service each different PE. Preferably, the CDCP pipeline 400 is configured to process medical claims for N number of PEs, with at least a portion of the CDCP pipeline 400 being implemented in a distributed manner via the CCS system 110 and one or more additional computer systems coupled to network 100 such as PE manager computing systems 180.

As discussed above, a medical claim can include medical claim data including, for instance, a claimant-specific member identifier, a claim-specific identifier (also referred to herein as a medical claim identifier), a PE identifier associated with the medical claim, diagnosis identifier/code, date(s) of service, provider identifier, and one or more line items. The one or more line items can comprise example, one or more codes indicating provider-provided medical services, pharmaceuticals, dosing amounts, treatment duration, and/or implant devices. The input stage 402 may optionally be configured to convert the “raw” data of the received medical claims into a standardized format, e.g., XML, JSON or other suitable format.

In any such cases, the input stage 402 then provides the received medical claims to the rule triggering stage 404. As shown, the rule triggering stage 404 can access a rule trigger DB 435, with rule trigger DB 435 being provided by storage components 130 (FIG. 1). Note, the rule trigger DB 435 may also be implemented in PE settings DB 434, or any other suitable storage location accessible via network 100. Likewise, rules DB 432 (also referred to as a medical claims rule DB) and the PE settings DB 434 may be provided by, for instance, med/pharma rules DB 132 and PE settings DB 134, respectively, of the storage components 130 (See FIG. 1).

In any event, the rule trigger DB 435 includes at least a lookup table that associates each PE of the plurality of PEs 490 with one or more rule triggers. The rule trigger DB 435 may then further associate each of the one or more rule triggers with a rule identifier for a rule stored in the rules DB 432. Each rule trigger can be configured to identify one or more target characteristics within a medical claim for additional analyzing via subsequent stages of the CDCP pipeline 400. For example, one non-limiting rule trigger can be configured to detect medical claims that include one or more line items directed to specific pharmaceutical drugs. Note. each PE can selectively enable N number of such rule triggers and provide one or more criteria to customize each enabled rule trigger.

The rule triggering stage 404 may then select one or more rule triggers based on querying the lookup of the rule trigger DB 435 using the PE identifier associated with the received medical claim(s). In scenarios where the CDCP pipeline 400 is configured to process claims associated with multiple different PEs, the rule triggering stage 404 may perform a plurality of such lookups either sequentially and/or in parallel when processing medical claims in a batch mode (e.g., when processing two or more medical claims received at the same type or otherwise provided in combination to the input stage 402) to identify rule triggers for the PEs.

The rule triggering stage 404 then identifies the presence of a rule triggering characteristic in the received medical claim. In response to the rule triggering characteristic(s) being present, the rule triggering stage 404 outputs one or more rule identifiers to the rule instantiation stage 406. Stated differently, each selected rule trigger is associated with one or more rule identifiers as discussed above, and in the event the rule triggering characteristic(s) associated with a given selected rule trigger is identified within a medical claim, the rule triggering stage 404 outputs at least the rule identifier(s) associated with the given selected rule trigger.

The rule instantiation stage 406 then receives the rule identifier(s) from the rule trigger stage 404. As further shown in FIG. 4, each PE can be associated with one or more enabled claim rules, e.g., based on a lookup table in the PE settings DB 434 or other suitable data store/database coupled the network 100. Each claim rule for a given PE may also be associated with one or more PE-provided rule parameters. For example, the PE settings DB 434 can include a lookup table that associates each PE with one or more claim rules as well as one or more PE-provided rule parameters for the one or more claim rules.

Some non-limiting example claim rules include a claim rule to detect one or more medically-unnecessary line items within a received medical claim. The medically-unnecessary line items may be identified based on, for instance, a diagnosis code within the claim rule being in conflict with a prescribed treatment. For example, a medical claim with a diagnosis code indicating a sleep apnea and a line item directed to a pharmaceutical drug for the treatment of blood pressure may therefore be identified as medically-questionable/unnecessary. Other such examples of potentially medically-unnecessary line items include, for instance, a medical claim for a female claimant/patient that includes a line item directed to pharmaceutical drug to manage prostate growth.

Additional non-limiting example claim rules include claim rules to detect dosing of a particular drug that exceeds a predetermined amount. Still further, additional claim rules include rules to detect orphan drug line items (e.g., drugs that are significantly more expensive than other drugs and generally limited to treatment of rare/uncommon conditions afflicting a small overall percentage of the population, e.g., less than 20% of the population) and/or off-label use line items (e.g., drugs with FDA approved indicators being used for non-approved conditions/disease).

Still further, additional non-limiting example claim rules include claim rules to detect catastrophic diagnosis line items, e.g., diagnosis that statistically increase the probability of requiring the use of stop loss insurance by a PE, and medical implant line items.

The PE-provided rule parameters 442 can be provided by users of the CCS system 110, such as by the users of the PE manager computing systems 180. The PE-provided rule parameters 442 may be utilized in combination with the aforementioned claim rules, and more particularly the predetermined rule logic 440 of each rule, to allow for fine-grain customization of rule logic. For example, the PE-provided rule parameters 442 can be utilized to provide one or more target drugs for use by claim rules directed to detecting particular orphan pharmaceuticals and/or off-label usage of pharmaceuticals. Note, the CCS system 110 can provide default/predetermined rule parameters that may be used in combination with the PE-provided rule parameters, and/or may be superseded/replaced by the PE-provided rule parameters 442.

Continuing on, the rule instantiation stage 406 then instantiates logic for the one or more claim rules, such as instantiated rule logic 411, e.g., based on the claim rule identifiers output by the rule trigger stage 404. The instantiated rule logic 411 can comprise, for instance, compiled and/or interpreted code/routines to allow for run-time execution. The instantiated logic can comprise, for example, logical condition statements (e.g., IF, ELSE IF, and other similar statements), logical comparisons (e.g., AND, OR, less than, equal to, less than or equal to, greater than or equal to and so on). In addition, the instantiated rule logic 411 can include instructions (e.g., for execution by the claim rule processing stage 408) to instantiate variables in memory and set the instantiated variables with data from queries to the Medical/Pharmaceutical DB 132, for example. Further, such instantiated rule logic 411 can be utilized to select, initialize, and/or interact with medical models that utilize input features such as procedure and diagnosis code combinations to identify claim abnormalities. Preferably, the medical models are so-called trainable medical models that can utilize information (also referred to as features) from various medical data stores, pharmaceutical databases, claimant records, and other sources of relevant data to allow the rule logic 411 to implement machine-learning techniques, and in some cases, utilize medical claim processing output by pipeline 400 as a feedback loop to continue training the medical models and further increase accuracy of abnormality detection.

Thus, the instantiated logic may comprise a relatively small number of relatively simple comparisons, e.g., such as to detect that a line item for a particular pharmaceutical and the associated line item for the dosing (which may be represented as a single medical code depending on the medical coding utilized) match based on a query to a pharmaceutical database that stores predefined dosage recommendations for a number of pharmaceutical drugs. Note, the predefined dosage may also be determined by querying the claimant DB 489 or other data store with one or more medical claims stored therein, with the predetermined dosage being therefore determined based on, for example, an average of the related dosage line items within the stored medical claims and/or derived via other algorithmic and/or statistic approaches based on the stored medical claim data with related dosage line items.

In some scenarios, the instantiated logic may comprise a relatively complex sequence of instructions to query multiple databases via network 100, for example, to perform a number of conditional checks and comparisons to detect a particular abnormality. For example, the instantiated logic may be configured to detect an inappropriate medical treatment/pharmaceutical based on querying the claimant information DB 189 and determining the prerequisite treatments were satisfied or unsatisfied as the case may be. For instance, some treatments such as chemotherapy require that certain pharmaceutical drugs be administered before others in a particular sequence. The instantiated rule logic 411 may therefore uncover/detect such latent abnormalities in medical records that would otherwise be undetected.

The claim rule processing stage 408 then utilizes the above-discussed instantiated rule logic 411 to detect medical claims of interest, e.g., medical claim of interest 499, for secondary review by the associated PE, for example. As shown, the claim rule processing stage 408 can utilize the instantiated rule logic 411 in combination with one or more data stores/databases, e.g., Medical/Pharmaceutical DB 480, claimant DB 489, to detect one or more predetermined claim abnormalities within the medical claim as discussed above.

The medical claim flagging and output stage 410 then flags the medical claim as a medical claim of interest based on the instantiated rule logic 411 detecting the one or more predetermined claim abnormalities. This can include the medical claim flagging and output stage 410 storing in indicator representing the medical claim of interest 499 in a memory, e.g., in a datastore/DB. The memory storing the indicators of medical claims of interest may then subsequently be accessed via, for example, requests to API 124 by authorized users of the CCS system 110.

Alternatively, or in addition, the medical claim flagging and output stage 410 can broadcast or otherwise transmit a message comprising at least one indicator of a medical claim of interest to a computing device of the associated PE, e.g., such as one or more of the PE manager computing systems 180. To this end, the CCS system 110 may utilize the PE settings DB 434 to identify the particular computing system associated with the PE (e.g., by IP, email address, or other suitable identifier). The PE manager computing systems 180 may then receive the event, and in response to receiving the same, place indicator(s) (e.g., visualized by a user interface) representing the medical claims of interest into a work item queue for secondary review of claims.

In any such cases, the medical claim flagging and output stage 410 may further also generate data representing the reason(s) for the medical claim being flagged including, for example, the particular rule(s) that detected an abnormality, one or more line items of the medical claim that were found to be abnormal by the particular rule(s), and the particular reason why the one or more line items were found to be abnormal and thus flagged (e.g., improper dosage, improper treatment duration, diagnosis and prescribed therapy mismatch, orphan drug, off-label usage of a pharmaceutical).

The medical claim flagging and output stage 410 may also further generate one or more suggested corrective actions such as modifying dosage, recommending a relatively less expensive implant option than what was specified within the medical claim, and requesting that an associated provider review the medical claim for correction and/or clarification.

In any such cases, the data representing the reasoning and/or suggested correction action may be stored with the associated medical claim of interest for use during a subsequent secondary review by a PE, for example. The claims of interest and corresponding optional additional data discussed above including reasoning and/or suggested corrective actions may be stored in association with an identifier of the PE. Users of the PE may then access the data store/DB to perform secondary review of medical claims of interest, to review the reasoning and/or corrective actions associated with the medical claims during secondary review, and to disposition/adjudicate the claims accordingly.

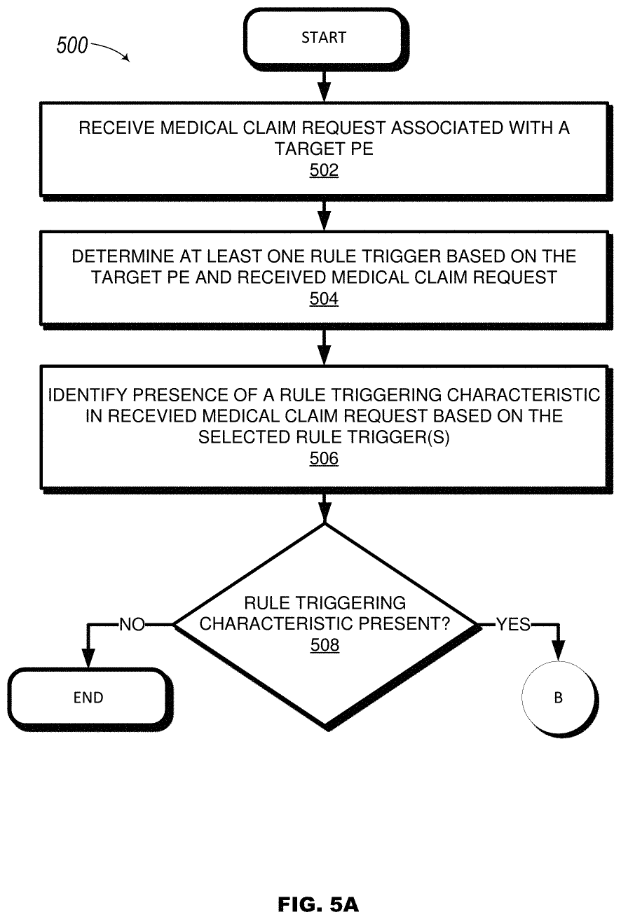

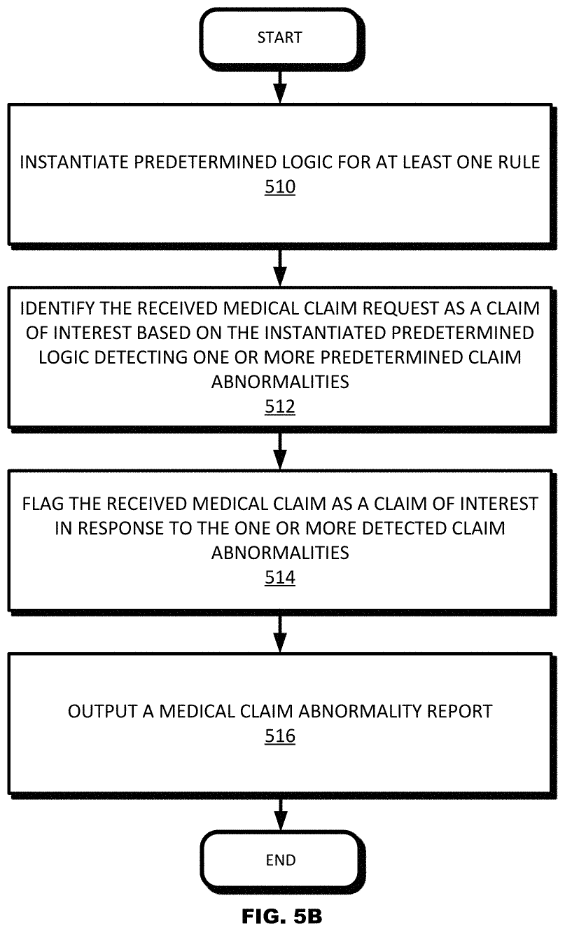

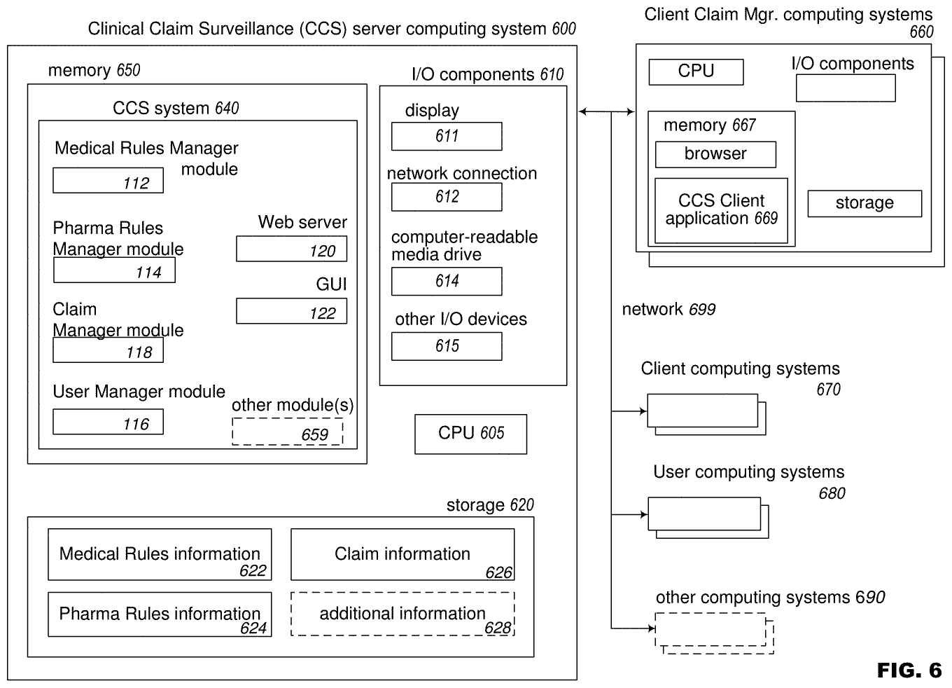

FIGS. 5A-5B collectively show an example process 500 that exemplifies various aspects and features of the present disclosure. Example process 500 may be implemented at least in part by a controller of the CCS system 110 such as CPU 605 discussed below with reference to FIG. 6, and preferably a controller implementing the CDCP pipeline such as CDCP pipeline 400 as discussed above. Example process 500 begins in act 502.

In act 502, the controller receives a medical claim request associated with a target PE. In act 504, the controller determines at least one rule trigger based on the target PE and the received medical claim request. In act 506, the controller identifies the presence of a rule trigger characteristic in the received medical claim request based on the selected rule trigger(s).

In act 508, the controller determines if at least one rule trigger characteristic is present based on the selected rule trigger(s). If the at least one rule trigger characteristic is present, the process 500 continues to act 510. Otherwise, the process 500 ends.

In act 510, the controller instantiates the predetermined logic for at least one claim rule. As discussed above, each rule trigger can be associated with one or more claim rules. Accordingly, the controller instantiates the predetermined logic according to the rule trigger and claim rule associations. Notably, this results in potentially a subset of claim rules being applied to a particular medical claim. Alternatively, or in addition, the controller can apply all (or a selected number) of claim rules without utilizing the rule triggers as a precondition/trigger.

In act 512, the controller identifies the received medical claim request as a claim of interest based on the instantiated predetermined logic detecting one or more predetermined claim abnormalities. In act 514, the controller flags the received medical claim as a claim of interest in response to the one or more detected claim abnormalities. In act 516, the controller optionally outputs a claim abnormality report, with the claim abnormality report providing the above-discussed flagging reasons and/or suggested corrective actions. The claim abnormality report may be stored with a representation of the flagged medical claim in a memory to, for instance, allow for subsequent retrieval by users of the CCS system 110.

FIG. 6 is a block diagram illustrating component-level functionality provided by a plurality of electronic circuits that, when in combined operation, are suitable for performing and configured to perform at least some of the techniques described herein. In the particular embodiment depicted, the plurality of electronic circuits is at least partially housed within a Clinical Claim Surveillance server computing system 600 executing an embodiment of a CCS system 640. The server computing system 600 includes one or more central processing units (“CPU”) or other processors 605 (also referred to as controllers), various input/output (“I/O”) components 610, storage 620, and memory 650, with the illustrated I/O components including a display 611, a network connection 612, a computer-readable media drive 614, and other I/O devices 615 (e.g., keyboards, mice or other pointing devices, microphones, speakers, GPS receivers, etc.). The server computing system 600 and CCS system 640 may communicate with other computing systems via one or more networks 699 (e.g., the Internet, one or more cellular telephone networks, etc.), such as Client Claim Manager computing systems 660, client computing systems 670, user computing systems 680, and other computing systems 690. Some or all of the other computing systems may similarly include some or all of the types of components illustrated for server computing system 600 (e.g., to have a CCS client application 669 executing in memory 667 of an originator computing system 660 in a manner analogous to CCS system 640 in memory 650).

In the illustrated embodiment, an embodiment of the CCS system 640 executes in memory 650 in order to perform at least some of the described techniques, such as by using the processor(s) 605 to execute software instructions of the system 640 in a manner that configures the processor(s) 605 and computing system 600 to perform automated operations implementing those described techniques. As part of such automated operations, the system 640 and/or other optional programs or modules 659 executing in memory 650 may store and/or retrieve various types of data, including in the example database data structures of storage 620. In this example, the data used may include various types of medical rules information in database (“DB”) 622, pharmaceutical rules information in DB 624, claim information in DB 626, and/or various types of other additional information in DB 628, such as various regulatory or policy information related to one or more client users and/or other users of the CCS system.

It will be appreciated that computing system 600 and computing systems 660, 670, 680 and 690 are merely illustrative, and are not intended to limit the scope of the present disclosure. The systems may instead each include multiple interacting computing systems or devices, and may be connected to other devices that are not specifically illustrated, including through one or more networks such as the Internet, via the Web, or via private networks (e.g., mobile communication networks, etc.). More generally, a device or other computing system may comprise any combination of hardware that may interact and perform the described types of functionality, optionally when programmed or otherwise configured with particular software instructions and/or data structures, including without limitation desktop or other computers (e.g., tablets, slates, etc.), database servers, network storage devices and other network devices, smart phones and other cell phones, consumer electronics, wearable and other user tracking devices, biometric monitoring devices, digital music player devices, handheld gaming devices, PDAs, wireless phones, pagers, electronic organizers, Internet appliances, television-based systems (e.g., using set-top boxes and/or personal/digital video recorders), and various other consumer and/or commercial products that include appropriate communication capabilities. In addition, the functionality provided by the illustrated CCS system 640 may in some embodiments be distributed in various modules. Similarly, in some embodiments, some of the functionality of the CCS system 640 may not be provided and/or other additional functionality may be available. When so arranged as described herein, each computing device may be transformed from a generic and unspecific computing device to a combination device comprising hardware and software configured for a specific and particular purpose.

It will also be appreciated that, while various items are illustrated as being stored in memory or on storage while being used, these items or portions of them may be transferred between memory and other storage devices for purposes of memory management and data integrity. Alternatively, in other embodiments some or all of the software modules and/or systems may execute in memory on another device and communicate with the illustrated computing systems via inter-computer communication. Thus, in some embodiments, some or all of the described techniques may be performed by hardware means that include one or more processors and/or memory and/or storage when configured by one or more software programs (e.g., the CCS system 640, and/or CCS client application software executing on computing systems 660, 670, 680 and/or 690) and/or data structures, such as by execution of software instructions of the one or more software programs and/or by storage of such software instructions and/or data structures. Furthermore, in some embodiments, some or all of the systems and/or modules may be implemented or provided in other manners, such as by consisting of one or more means that are implemented at least partially in firmware and/or hardware (e.g., rather than as a means implemented in whole or in part by software instructions that configure a particular CPU or other processor), including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc. Some or all of the modules, systems and data structures may also be stored (e.g., as software instructions or structured data) on a non-transitory computer-readable storage mediums, such as a hard disk or flash drive or other non-volatile storage device, volatile or non-volatile memory (e.g., RAM or flash RAM), a network storage device, or a portable media article (e.g., a DVD disk, a trusted electronic compliance document disk, an optical disk, a flash memory device, etc.) to be read by an appropriate drive or via an appropriate connection. The systems, modules and data structures may also in some embodiments be transmitted via generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of the present disclosure may be practiced with other computer system configurations.

The descriptions of the various techniques disclosed herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments and techniques disclosed herein.

It will be appreciated that in some embodiments the functionality provided by routines discussed above may be provided in alternative ways, such as being split among more routines or consolidated into fewer routines. Similarly, in some embodiments illustrated routines may provide more or less functionality than is described, such as when other illustrated routines instead lack or include such functionality respectively, or when the amount of functionality that is provided is altered. In addition, while various operations may be illustrated as being performed in a particular manner (e.g., in serial or in parallel) and/or in a particular order, it will be appreciated that in other embodiments the operations may be performed in other orders and in other manners. It will also be appreciated that particular data structures discussed above may be structured in different manners, such as by having a single data structure split into multiple data structures or by having multiple data structures consolidated into a single data structure. Similarly, in some embodiments, illustrated data structures may store more or less information than is described, such as when other illustrated data structures instead lack or include such information respectively, or when the amount or types of information that is stored is altered.

The present disclosure may be implemented in a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (compliance document-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions. Such computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform operations related to the described functionality, or carry out combinations of special purpose hardware and computer instructions.

### Additional Examples and Architecture

Example 1 is a system for processing medical claims from a plurality of provider entities to identify medical claims of interest for secondary review by an associated payer entity (PE), the system comprising a controller communicatively coupled to a computer network and configured to receive a medical claim request via the computer network, the received medical claim request being associated with a target PE, select at least a first claim rule from a plurality of claim rules based at least in part on the target PE and the received medical claim request, the first claim rule including predetermined logic to detect a first predetermined claim abnormality, identify the received medical claim request as a claim of interest based on instantiating the predetermined logic of the first claim rule and detecting the first predetermined abnormality as present in the received medical claim, and store an indicator of the received medical claim request in a memory in response to identifying the received medical claim request as a claim of interest.

Example 2 includes the features of Example 1, and wherein the system further comprises a medical claim rules database, and wherein the controller is further configured to select the first claim rule based on querying a first lookup table that associates an identifier of the target PE with one or more identifiers of claim rules stored in the medical claim rules database.

Example 3 includes the features of Example 2, and wherein the system further comprises a PE settings database, the PE settings database storing a representation of at least one PE-provided claim rule parameter, each PE-provided claim rule parameter being configured to be utilized by predetermined logic of an associated claim rule to detect at least one predetermined abnormality in a given medical claim.

Example 4 includes the features of any one of Examples 2-3, wherein the controller is further configured to select the first claim rule based on querying a second lookup table in the PE settings database that associates an identifier of the target PE with the at least one PE-provided claim rule parameter.

Example 5 includes the features of Example 4, wherein the controller is further configured to identify the received medical claim request as a claim of interest based on instantiating the predetermined logic of the first claim rule, the predetermined logic configured to detect the predetermined abnormality as present in the received medical claim based at least in part on the at least one PE-provided claim rule parameter.

Example 6 includes the features of any one of Examples 3-5, wherein the system includes a plurality of predetermined PEs including the target PE, each of the plurality of predetermined PEs having an associated PE identifier stored within the PE settings database and a plurality of associated PE-provided claim rule parameters.

Example 7 includes the features of any one of Examples 3-6, wherein the PE settings database further includes a claim rule enabled value indicating whether each of the one or more claim rules associated with a given PE is enabled or disabled.

Example 8 includes the features of any one of Examples 1-7, wherein the first selected claim rule includes logic to cause the controller to identify whether the received medical claim request includes a line item identifying one or more target pharmaceutical drugs.

Example 9 includes the features of Example 8, wherein the first selected claim rule includes logic to cause the controller to identify whether the received medical claim request includes a line item that indicates a dosage amount for the one or more target pharmaceutical drug exceeds a predefined dosage amount.

Example 10 includes the features of Example 9, further comprising a memory to store at least a first claim rule parameter associated with the target PE, wherein the one or more target pharmaceutical drugs are defined by the first claim rule parameter, and wherein the controller is further configured to instantiate the predetermined logic of the first selected claim rule based at least in part on retrieving the first claim rule parameter from the memory.

Example 11 includes the features of any one of Examples 9-10, wherein the predefined dosage amount is defined within a second claim rule parameter stored in a memory, the second claim rule parameter being associated with the target PE, and wherein the controller is further configured to instantiate the logic of the first selected claim rule based at least in part on retrieving the second claim rule parameter from the memory.

Example 12 includes the features of any one of Examples 9-11, wherein the controller is further configured to determine the predefined dosage amount based on querying a pharmaceutical database.

Example 13 includes the features of any one of Examples 9-12, further comprising a medical claims database to store a plurality of received medical claims, wherein the controller is further configured to determine the predefined dosage amount based at least in part on identifying one or more medical claims stored in the medical claims database that include line items identifying the one or more target pharmaceutical drugs and associated dosage amounts.

Example 14 includes the features of any one of Examples 1-13, wherein the first selected claim rule includes logic to cause the controller identify whether the received medical claim request includes a line item identifying one or more target pharmaceutical drugs, and logic to further cause the controller to determine if the one or more identified target pharmaceutical drugs include an off-label treatment indicator.

Example 15 includes the features of Example 14, wherein the controller is further configured to determine if the one or more identified target pharmaceutical drugs include an off-label treatment indicator based at least in part on a querying a pharmaceutical database and a diagnosis code within the received medical claim request.

Example 16 includes the features of any one of Examples 1-15, wherein the first selected claim rule includes logic to cause the controller identify whether the received medical claim request includes a line item identifying at least one orphan pharmaceutical drug.

Example 17 includes the features of any one of Examples 1-16, wherein the controller is further configured to identify the received medical claim request as a claim of interest based on converting the received medical request or at least a portion thereof into a standardized format.

Example 18 includes the features of any one of Examples 1-17, wherein the controller is further configured to, in response to identifying the received medical claim request as a claim of interest, send a message including an indicator of the received medical claim request to a remote computing device via the computer network.

Example 19 includes the features of any one of Examples 1-18, wherein the memory provides a list of claims of interest for secondary review of medical claims.

Example 20 includes the features of Example 19, further comprising a web server or application programming interface (API) to receive requests from a remote computing device via the computer network to read and/or update the list of claims of interest for secondary review stored in the memory.

Example 21 includes the features of any one of Examples 1-20, a medical claims rule database to store the at least one selected claim rule, and a web server or application programming interface (API) to receive a request to create, update, or delete one or more claim rules stored in the medical claims rule database.

Example 22 is a computer-implemented method for processing medical claims from a plurality of provider entities to identify medical claims of interest for secondary review by an associated payer entity (PE), the method comprising receiving a medical claim request via a computer network, the received medical claim request being associated with a target PE, selecting at least a first claim rule from a plurality of claim rules based at least in part on the target PE and the received medical claim request, the first claim rule including predetermined logic to detect a first predetermined claim abnormality, identifying the received medical claim request as a claim of interest based on instantiating the predetermined logic of the first claim rule and detecting the first predetermined abnormality as present in the received medical claim, and storing an indicator of the received medical claim request in a memory in response to identifying the received medical claim request as a claim of interest.

Example 23 includes the features Example 22, further comprising selecting the first claim rule based on querying a first lookup table that associates an identifier of the target PE with one or more identifiers of claim rules stored in a medical claim rules database.

Example 24 includes the features of any one of Examples 21-23, wherein selecting the first claim rule further comprising querying a second lookup table in a PE settings database that associates an identifier of the target PE with at least one PE-provided claim rule parameter.

Example 25 includes the features of Example 24, wherein identifying the received medical claim request as a claim of interest further comprises instantiating the predetermined logic of the first claim rule, the predetermined logic configured to detect the predetermined abnormality as present in the received medical claim based at least in part on the at least one PE-provided claim rule parameter.

Example 26 includes the features of any one of Examples 22-25, wherein identifying the received medical claim request as a claim of interest further comprises identifying that a line item of the received medical claim request includes an identifier of one or more target pharmaceutical drugs.

Example 27 includes the features of any one of Examples 22-26, wherein identifying the received medical claim request as a claim of interest further comprises identifying that a line item of the received medical claim request includes a dosage amount identifier that exceeds a predefined dosage amount.

Example 28 includes the features of any one of Examples 22-27, wherein identifying the received medical claim as a claim of interest further comprises identifying whether the received medical claim request includes a line item identifying one or more target pharmaceutical drugs for use in an off-label manner.

Example 29 includes the features of any one of Examples 22-28, wherein identifying the received medical claim as a claim of interest further comprises identifying whether the received medical claim request includes a line item identifying one or more orphan pharmaceutical drugs.

Example 30 includes the features of any one of Examples 22-29, wherein identifying the received medical claim request as a claim of interest further comprises converting the received medical request or at least a portion thereof into a standardized format.

Example 31 includes the features of any one of Examples 21-30, further comprising, in response to identifying the received medical claim request as a claim of interest, sending a message including an indicator of the received medical claim request to a remote computing device via a computer network.

From the foregoing it will be appreciated that, although specific embodiments have been described herein for purposes of illustration, various modifications may be made without deviating from the spirit and scope of the disclosure described herein. Accordingly, the disclosure is not limited except as by corresponding claims and the elements recited by those claims. In addition, while certain aspects of the disclosure may be presented in certain claim forms at certain times, the inventors contemplate the various aspects of the disclosure in any available claim form. For example, while only some aspects of the disclosure may be recited as being embodied in a computer-readable medium at particular times, other aspects may likewise be so embodied.

What is claimed is:

1. A system for processing medical claims from a plurality of provider entities to identify medical claims of interest for secondary review by an associated payer entity (PE), the system comprising:
a controller communicatively coupled to a computer network and configured to:
receive a medical claim request via the computer network, the received medical claim request being associated with a target PE;
select at least a first claim rule from a plurality of claim rules based at least in part on the target PE and the received medical claim request, the first claim rule including predetermined logic to detect a first predetermined claim abnormality;
identify the received medical claim request as a claim of interest based on instantiating the predetermined logic of the first claim rule and detecting the first predetermined abnormality as present in the received medical claim; and
store an indicator of the received medical claim request in a memory in response to identifying the received medical claim request as a claim of interest.

2. The system of claim 1, wherein the system further comprises a medical claim rules database, and wherein the controller is further configured to:
select the first claim rule based on querying a first lookup table that associates an identifier of the target PE with one or more identifiers of claim rules stored in the medical claim rules database.

3. The system of claim 2, wherein the system further comprises a PE settings database, the PE settings database storing a representation of at least one PE-provided claim rule parameter, each PE-provided claim rule parameter being configured to be utilized by predetermined logic of an associated claim rule to detect at least one predetermined abnormality in a given medical claim.

4. The system of claim 3, wherein the controller is further configured to select the first claim rule based on querying a second lookup table in the PE settings database that associates an identifier of the target PE with the at least one PE-provided claim rule parameter.

5. The system of claim 4, wherein the controller is further configured to identify the received medical claim request as a claim of interest based on instantiating the predetermined logic of the first claim rule, the predetermined logic configured to detect the predetermined abnormality as present in the received medical claim based at least in part on the at least one PE-provided claim rule parameter.

6. The system of claim 1, wherein the first selected claim rule includes logic to cause the controller to identify whether the received medical claim request includes a line item identifying one or more target pharmaceutical drugs.

7. The system of claim 6, wherein the first selected claim rule includes logic to cause the controller to identify whether the received medical claim request includes a line item that indicates a dosage amount for the one or more target pharmaceutical drug exceeds a predefined dosage amount.

8. The system of claim 1, wherein the first selected claim rule includes logic to cause the controller to identify whether the received medical claim request includes a line item identifying at least one orphan pharmaceutical drug.

9. The system of claim 1, wherein the controller is further configured to, in response to identifying the received medical claim request as a claim of interest, send a message including an indicator of the received medical claim request to a remote computing device via the computer network.

10. The system of claim 1, wherein the memory provides a list of claims of interest for secondary review of medical claims, and wherein the system further comprises a web server or application programming interface (API) to receive requests from a remote computing device via the computer network to read and/or update the list of claims of interest for secondary review stored in the memory.

11. A computer-implemented method for processing medical claims from a plurality of provider entities to identify medical claims of interest for secondary review by an associated payer entity (PE), the method comprising:
receiving a medical claim request via a computer network, the received medical claim request being associated with a target PE;
selecting at least a first claim rule from a plurality of claim rules based at least in part on the target PE and the received medical claim request, the first claim rule including predetermined logic to detect a first predetermined claim abnormality;
identifying the received medical claim request as a claim of interest based on instantiating the predetermined logic of the first claim rule and detecting the first predetermined abnormality as present in the received medical claim; and
storing an indicator of the received medical claim request in a memory in response to identifying the received medical claim request as a claim of interest.

12. The computer-implemented of claim 11, further comprising selecting the first claim rule based on querying a first lookup table that associates an identifier of the target PE with one or more identifiers of claim rules stored in a medical claim rules database.

13. The computer-implemented of claim 11, wherein selecting the first claim rule further comprising querying a second lookup table in a PE settings database that associates an identifier of the target PE with at least one PE-provided claim rule parameter.

14. The computer-implemented of claim 13, wherein identifying the received medical claim request as a claim of interest further comprises instantiating the predetermined logic of the first claim rule, the predetermined logic configured to detect the predetermined abnormality as present in the received medical claim based at least in part on the at least one PE-provided claim rule parameter.

15. The computer-implemented of claim 11, wherein identifying the received medical claim request as a claim of interest further comprises identifying that a line item of the received medical claim request includes an identifier of one or more target pharmaceutical drugs.

16. The computer-implemented of claim 11, wherein identifying the received medical claim request as a claim of interest further comprises identifying that a line item of the received medical claim request includes a dosage amount identifier that exceeds a predefined dosage amount.

17. The computer-implemented of claim 11, wherein identifying the received medical claim as a claim of interest further comprises identifying whether the received medical claim request includes a line item identifying one or more target pharmaceutical drugs for use in an off-label manner.

18. The computer-implemented of claim 11, wherein identifying the received medical claim as a claim of interest further comprises identifying whether the received medical claim request includes a line item identifying one or more orphan pharmaceutical drugs.

19. The computer-implemented of claim 11, wherein identifying the received medical claim request as a claim of interest further comprises converting the received medical request or at least a portion thereof into a standardized format.

20. The computer-implemented of claim 11, further comprising, in response to identifying the received medical claim request as a claim of interest, sending a message including an indicator of the received medical claim request to a remote computing device via a computer network.
